(19) United States
(12) Patent Application Publication
(10) Pub. No.: US 2026/0167376 A1
(43) Pub. Date: Jun. 18, 2026

(54) STERILE CONTAINER TRAY AND STERILE CONTAINER COMPRISING A STERILE CONTAINER TRAY

(71) Applicant: Aesculap AG, Tuttlingen (DE)

(72) Inventors: Matthias Schweizer, Tuttlingen (DE); Stephan Dorau, Rottweil (DE); Thomas Hagen, Tuttlingen (DE); Christian Wenzler, Wurmlingen (DE); Joachim Amann, Muehlingen - Zoznegg (DE); Ruediger Sturm, Orsingen-Nenzingen (DE); Johannes Adam, Tuttlingen (DE)

(21) Appl. No.: 19/415,161

(22) Filed: Dec. 10, 2025

(30) Foreign Application Priority Data
Dec. 13, 2024 (DE) 10 2024 137 607.4

(51) Int. Cl. B65D 1/34; B65D 43/02; B65D 53/02

(52) U.S. Cl. CPC B65D 1/34; B65D 43/0218; B65D 53/02; B65D 2543/00194; B65D 2543/005; B65D 2543/00537; B65D 2543/00555; B65D 2543/00972

(57) ABSTRACT

A sterile container includes a sterile container tray. The sterile container tray can be closed by a sterile container lid. The sterile container tray has a tray bottom and a surrounding tray wall. The tray wall can run perpendicularly to the tray bottom. The sterile container tray can also have a tray rim forming a free end of the tray wall. The tray rim can include at least a first rim region and a second rim region. The first rim region can have a first height relative to the tray bottom, and the second rim region can have a second height relative to the tray bottom that is different from the first height.

### CROSS-REFERENCE TO RELATED APPLICATION

This application claims priority under 35 U.S.C. § 119 to German Application No. 10 2024 137 607.4, filed on Dec. 13, 2024, the content of which is incorporated by reference herein in its entirety.

### FIELD

The present disclosure relates to a sterile container tray of a sterile container, which can be closed by a sterile container lid. The sterile container tray comprises a tray bottom, a surrounding tray wall, in particular running perpendicular to the tray bottom, and a tray rim forming a free end of the tray wall.

Furthermore, the present disclosure relates to a sterile container comprising a sterile container tray and a sterile container lid for closing the sterile container tray. In a closed position, the sterile container tray and the sterile container lid define a container interior.

### BACKGROUND

In particular, to provide a germ barrier, an in particular surrounding sealing element is provided which, in the closed position, lies against both the container tray as well as the container lid.

Sterile containers of the type described above are known in many different forms. They serve, in a well-known manner, in particular for the germ-free storage, for transport and for the provision of previously cleaned and sterilized surgical instruments and implants. Mainly sealing cords, sealing lips and similar sealing elements made of silicone, silicone foams and similar materials are used as sealing element.

Sterile containers made of metallic materials are usually equipped with a closing device to clamp the container lid against the container tray in a defined manner in the closed position. The sealing element can be compressed.

For the best possible seal between the container lid and container tray, a surrounding contact by the sealing element or even pressure by the sealing element on the container tray via the container lid is required. To achieve the most even pressure possible, it is advantageous to design the components to be as (flexurally) stiff as possible.

Sterile container lids are usually designed as flat bodies which are clamped onto the container tray, for example at the end faces, using the closing device. Due to the counterforce exerted by compression of the sealing element in the closed position, the relatively flat container lid may undergo elastic deformation or bending. In particular in the middle of one longitudinal side of the container lid, the sealing element is pressed less firmly so that a sufficient seal is no longer ensured. The larger or longer and the less rigid the container lid is designed, the greater the elastic bending of the container lid. The bending of the container lid can be only a few tenths of a millimeter, but also a few millimeters.

It is known from the prior art to compensate for the deformation of the lid by a special shape or design of the sealing element. Other sterile container systems provide for an increase in the rigidity of the container lid through stiffening ribs and stiffening contours. It is also known to compensate for the deformation of the lid by means of a special shape or pre-bending of the lid. For example, the lid can be pre-bent against the elastic deflection of the lid.

### SUMMARY

The object of the present disclosure is to provide a sterile container tray and a sterile container with improved sealing.

A sterile container tray of a sterile container, which can be closed by a sterile container lid, comprises a tray bottom, a surrounding tray wall, in particular running perpendicular to the tray bottom, and a tray rim forming a free end of the tray wall, in particular a surrounding rim. According to the present disclosure, the tray rim of the sterile container tray comprises at least a first and a second rim region, wherein the first and the second rim region each have a height, different from each other, relative to the tray bottom of the sterile container tray.

To compensate for the deformation or bending of the sterile container lid in the closed position and, in particular, to design the sterile container ideally tight in the closed position, a height-variable tray rim is proposed instead of a horizontally cut tray rim. The height of the tray rim therefore varies over the circumference of the tray rim.

The height variation of the tray rim described in the present application expressly refers to a change in the vertical position of the tray rim relative to the tray bottom along the circumference in the circumferential direction. This involves not merely a curvature or bending of the tray rim in a radial or tangential direction, e.g., by bulging or inclination as is known from the prior art, but an actual change in the height of the tray rim, in particular an upper tray rim, so that the tray rim, and in particular an upper edge of the tray rim, exhibits different heights in the circumferential direction.

The upper edge of the tray rim refers to the line or surface that extends along the uppermost boundary of the tray rim and represents the highest point in the vertical direction at any location along the circumference. A variation in the height of the upper edge in the circumferential direction exists when the vertical position of the uppermost boundary of the tray rim varies relative to the tray bottom. Purely form-related curvatures, bends or roundings, for example an outwardly curved rim, which do not represent a change in the upper edge of the tray rim in the circumferential direction, are not a height variation within the meaning of this definition.

In contrast to known embodiments with an outwardly or inwardly curved tray rim, in which an upper edge remains substantially at a constant height, the tray rim in accordance with the present disclosure is designed such that the height of the tray rim or an upper edge relative to the tray bottom varies along the circumference in accordance with the first rim region and the second rim region.

The container tray according to the present disclosure has a tray rim which has a height variation in the circumferential direction. This means that the vertical position of the upper edge of the tray rim relative to the tray bottom varies in portions along the circumference. This height variation does not lie in a single plane, neither horizontal nor inclined. In particular, the first and second rim regions, which each have a height, different from each other, relative to the tray bottom of the sterile container tray, are not in a common plane, neither horizontally nor inclined. In particular, the design according to the present disclosure differs from embodiments in which the tray rim is inclined or curved, but its upper edge runs continuously in a horizontal or inclined plane.

A height difference between the height in a region with the lowest height, i.e., a minimum region, of the tray rim and the height at a region with the greatest height, i.e., a maximum region, of the tray rim is, for example, between 0.1 mm and 8 mm, in particular between 0.3 mm and 5 mm, preferably between 0.5 mm and 3 mm.

In accordance with one embodiment, it is provided that the first rim region comprises a minimum region with the lowest height of the tray rim relative to the tray bottom, and the second rim region comprises a maximum region with the greatest height of the tray rim relative to the tray bottom. A minimum region and/or a maximum region can be a point, in the sense of a minimum point or maximum point, or a region with a certain longitudinal or transverse extent. In addition to the minimum region with the lowest height and/or the maximum region with the greatest height, further local minimum or maximum regions may be provided which define a minimum or a maximum relative to a surrounding region.

Advantageously, for example, it can be provided that at least two first rim regions and at least two second rim regions are provided. The tray rim then comprises, for example, two minimum regions and two maximum regions around the circumference of the tray rim.

In accordance with one embodiment, it is provided that the tray wall of the sterile container tray comprises two opposing end-face wall portions and two opposing longitudinal wall portions, wherein an end-face rim portion of the tray rim is formed on each end face, and a longitudinal rim portion of the tray rim is formed on each longitudinal side, and wherein a first rim region is formed at least partially in the end-face rim portions, and a second rim region is formed at least partially in the longitudinal rim portions. For example, a closing device for closing the sterile container tray with a corresponding sterile container lid, or at least parts of the closing device, is provided on the end-face wall portions, or a closing device engages with the end wall portions. The longitudinal wall portions can, but do not necessarily have to, comprise a greater extension than the end-face wall portions. Advantageously, the maximum regions of the tray rim are formed on the longitudinal rim portions, i.e., on the sides of the sterile container tray at which no closing device is provided or engages. The minimum regions of the tray rim are accordingly formed on the end-face rim portions, i.e., on the sides of the sterile container tray at which a closing device is provided or engages.

According to one embodiment, it can be provided that the first and/or the second rim region can be formed by a contour of the tray rim that is at least in portions straight relative to the tray bottom, and/or, in particular continuously, rising and/or, in particular continuously, falling, in particular bent or curved.

The contour of the tray rim can be at least in portions convex and/or concave. For example, the contour of the tray rim can be concave in the vicinity of a minimum region or in a minimum region. For example, the contour of the tray rim can be convex in the vicinity of a maximum region or within a maximum region. If the tray rim comprises further local minimum or maximum regions, the contour of the tray rim in the vicinity of these regions or in these regions can also be concave or convex.

According to one embodiment, it is provided that the tray wall comprises rounded corners, wherein a corner rim portion of the tray rim is formed at each of the corners, and wherein a first and/or a second rim region is formed at least partially in a corner rim portion. It is therefore proposed that a height variation of the tray rim is also formed in one or more corner rim portions.

According to one embodiment, it is provided that the tray rim is a cut tray rim, or wherein the tray rim comprises an in particular outwardly curved profile. In the case of a curved profile, the tray rim can comprise a rim surface that extends, in particular, approximately in the horizontal plane and/or in the vertical plane. In this case, the rim surface is designed with varying heights.

The sterile container tray, for example, is manufactured in a deep-drawing method. The height-varied geometry of the tray rim of the sterile container tray can be produced in a cutting method. In the case of a curved profile, the height-varying geometry of the tray rim can also be incorporated in a bending method or in a pressing method to produce the curved contour.

Further embodiments relate to a sterile container comprising a sterile container tray in accordance with the embodiments described above and comprising a sterile container lid for closing the sterile container tray in a closed position, and comprising a sealing element which, in the closed position, lies against both the sterile container tray as well as the sterile container lid. The sterile container lid may, in particular, comprise a side rim that extends beyond the sterile container tray. The sterile container comprises, for example, a closing device which is provided in particular on the sterile container tray and/or on the sterile container lid in order to clamp the sterile container lid in the closed position against the sterile container tray in a defined manner. The sealing element can be compressed. By means of the proposed height-varying contour of the tray rim, deformation or bending of the sterile container lid can be compensated for, and improved sealing can thereby be achieved. Further measures to compensate for the deformation of the sterile container lid can then be dispensed with.

According to one embodiment, it can be provided that the sealing element lies on the tray rim of the sterile container tray in the closed position, or that the sealing element lies against an outwardly facing region of the tray wall of the sterile container tray in the closed position.

If the sealing element lies against an outwardly facing region of the tray wall of the container tray in the closed position, it is advantageous if the sterile container lid comprises a side rim that overlaps the sterile container tray. In the closed position, the sealing element then lies against an inwardly directed region of the overlapping side rim of the sterile container lid. In this case, it can prove advantageous if a damping element is provided which lies on the tray rim of the sterile container tray in the closed position. Such a damping element is intended to prevent direct metal-to-metal contact between the sterile container tray and the sterile container lid. The damping element does not need to provide a seal.

### BRIEF DESCRIPTION OF THE DRAWINGS

Further advantages are found in the description and the attached drawings. Embodiments of the present disclosure are shown in the drawings and explained in more detail in the following description. In this case, the same reference signs in different drawings denote the same or at least functionally comparable elements. In the description of individual drawings, reference may also be made to elements from other drawings.

FIG. 1a)-1c) are different views of a sterile container tray in accordance with a first embodiment;

FIG. 2a)-2c) are different views of a sterile container tray in accordance with a further embodiment;

FIG. 3a)-3c) are different views of a sterile container tray in accordance with a further embodiment; and

FIGS. 4a) and 4b) are different views of a sterile container comprising a sterile container tray in accordance with an embodiment shown in any of the preceding Figures, with a sterile container lid and a sealing element.

### DETAILED DESCRIPTION

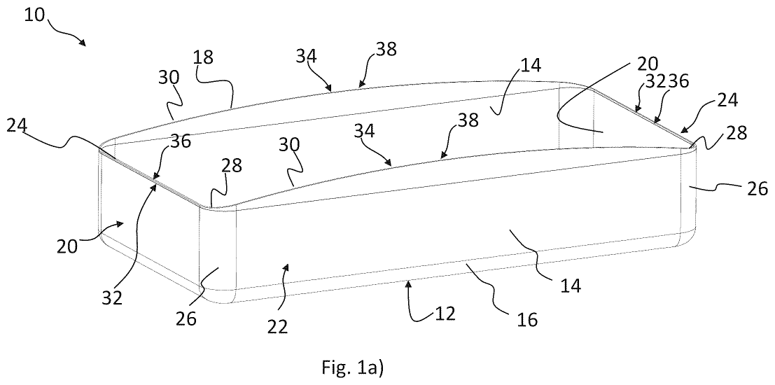

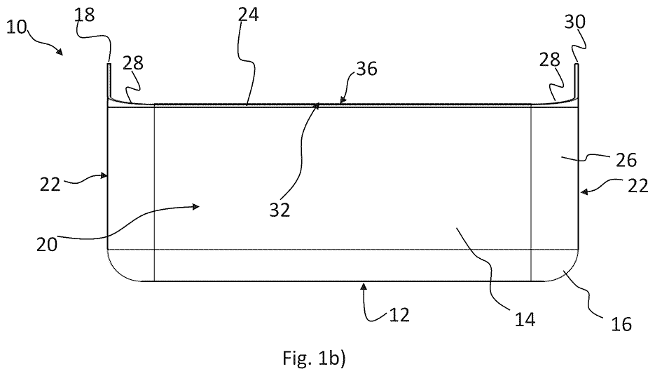

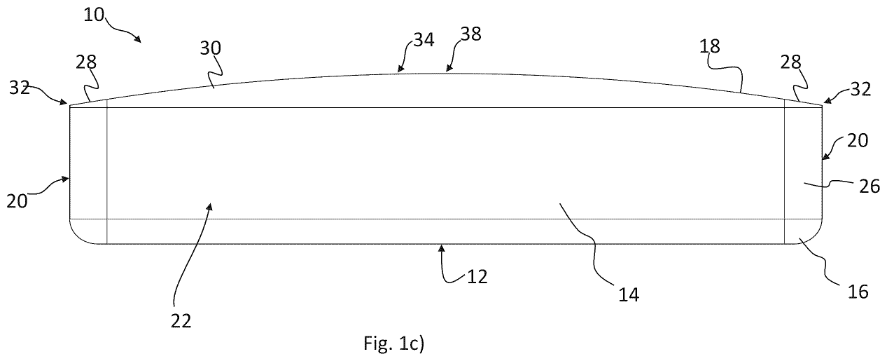

FIG. 1a) shows a sterile container tray 10 of a sterile container which can be closed by a sterile container lid (not shown). The sterile container tray 10 comprises a tray bottom 12 and a surrounding tray wall 14. The tray wall 14 generally runs perpendicular to the tray bottom 12, wherein in the example, a transition region 16 from the tray bottom 12 to tray wall 14 is rounded.

The sterile container tray 10 comprises a surrounding tray rim 18, which forms a free end of the tray wall 14. In the example, tray rim 18 is a cut tray rim. According to alternative embodiments (not shown), the tray rim 18 can comprise an in particular outwardly curved profile.

The tray wall 14 of the sterile container tray 10 comprises two opposing end-face wall portions 20 and two opposing longitudinal wall portions 22.

For example, a closing device (not shown) for closing the sterile container tray with a corresponding sterile container lid or at least parts of the closing device is provided on the end wall portions 20, or a closing device engages with the end-face wall portions 20. In the example, the longitudinal wall portions 22 comprise a greater extent than the end-face wall portions 20.

On each end-face wall portion 20, an end edge portion 28 of the tray rim 18 is formed, and on each longitudinal wall portion 22, a longitudinal edge cut 30 of the tray rim 18 is formed.

In the example, the tray wall 18 comprises rounded corners 26 at the transition from an end-face wall portion 20 to a longitudinal wall portion 22. At the rounded corners 26, a corner rim portion 28 of the tray rim 18 is formed.

In the example, the tray rim 18 comprises two first rim regions 32 and two second rim regions 34, wherein the first rim regions 32 and the second rim regions 34 each have a height, different from each other, relative to the tray bottom 12 of the sterile container tray 10.

In the example, a first rim region 32 is formed in the end-face rim portions 24 and a second rim region 34 is formed in the longitudinal rim portions 30.

A corresponding first rim region 32 comprises a minimum region 36 with the lowest height of the tray rim 18 relative to the tray bottom 12. In the example, the tray rim 18 in the first rim region 32 is formed with a straight horizontal contour. Accordingly, the minimum region 36 extends over the first rim region. In the example, the contour of the tray rim 18 in the end-face rim portion 24 is straight, cf. FIG. 1b). In the corner rim portion, the tray rim comprises a rising or falling contour.

A corresponding second rim region comprises a maximum region 38 with the greatest height of the tray rim 18 relative to the tray bottom 12. In the example, the tray rim 18 in the second rim region 34 on the longitudinal rim portion 30 is formed with a curved, in particular continuously, rising contour towards the center of the longitudinal rim portion 30. Therefore, the maximum region 38 in the example is a maximum point, i.e., the point with the greatest height of the tray rim 18.

In the example, the contour of the tray rim 18 in the longitudinal rim portion 26 is continuously convex, cf. FIG. 1c).

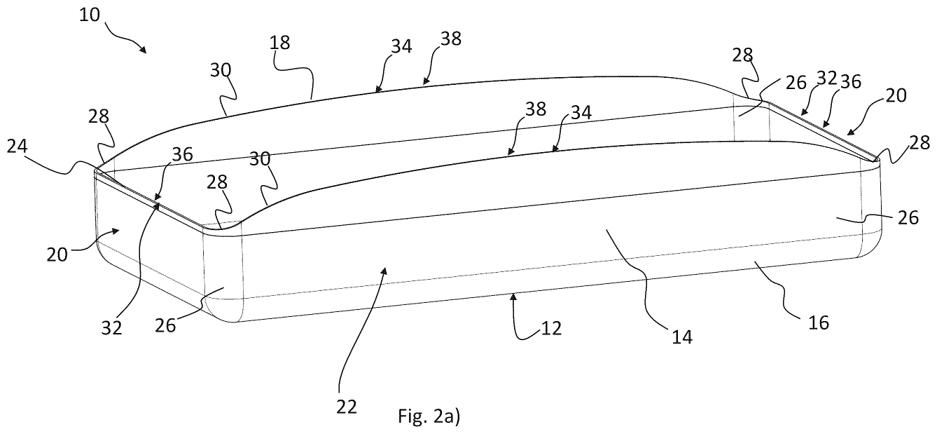

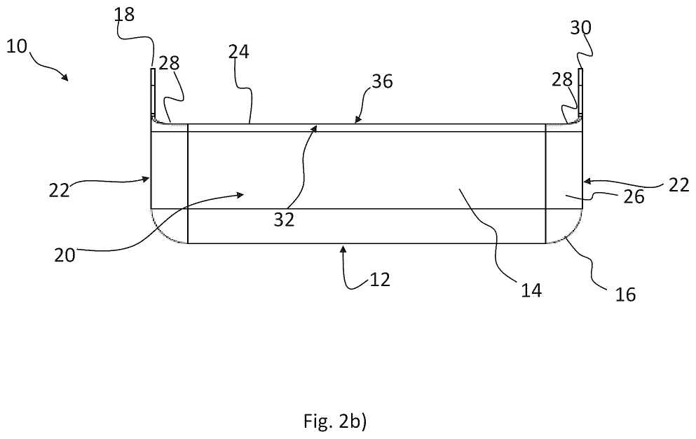

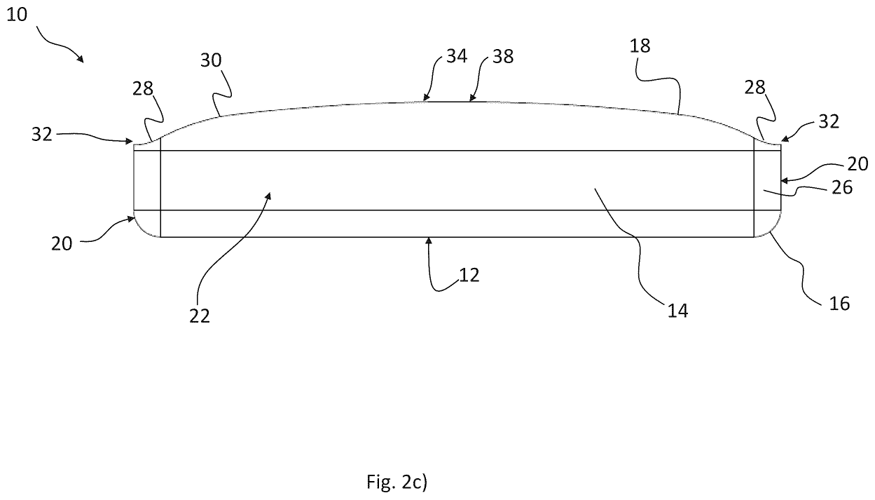

FIG. 2a)-2c) show different views of a sterile container tray 10 according to a further embodiment.

The sterile container tray 10 is designed similarly to the sterile container tray 10 shown in FIG. 1a)-1c). The sterile container trays 10 according to FIG. 1a)-2c) differ in the contour of the tray rim 18.

In the sterile container tray according to FIG. 2a)-2c), the contour of the tray rim 18 in the longitudinal rim portion 26 is alternately concave and convex, cf. FIG. 2a) and FIG. 2c). Accordingly, the contour of the tray rim takes the form of a double S-curve along the longitudinal rim portion 26. The longitudinal rim portion 26 has, in the center, the maximum region 38 with the greatest height of the tray rim 18 relative to the tray bottom 12. Starting from the center, the contour of the tray rim 18 in the longitudinal rim portion 26 is initially shaped convex and then transitions into a concave shape.

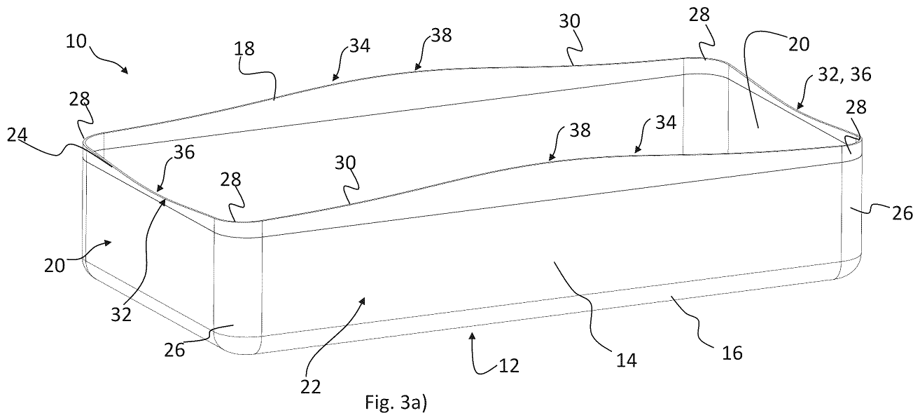

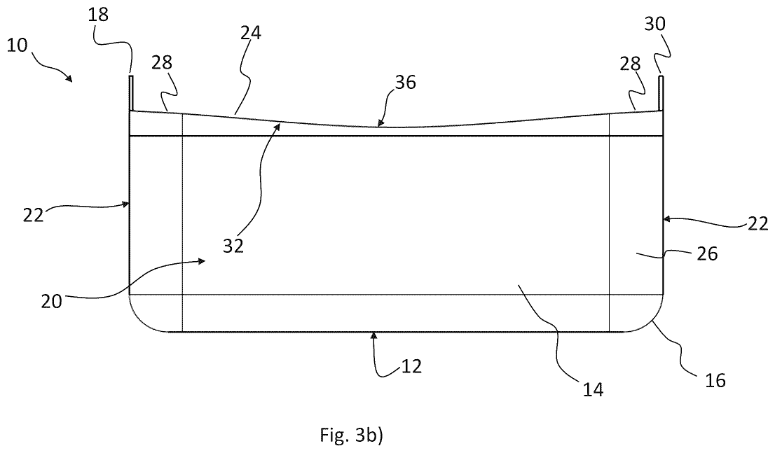

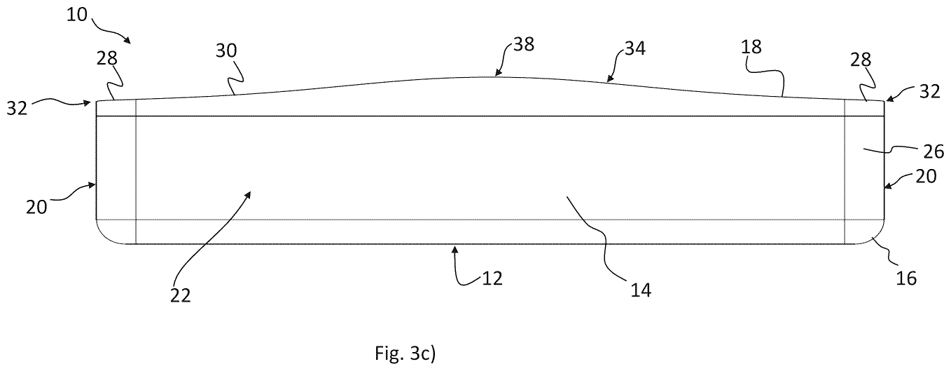

FIGS. 3a)-3c) Show Different Views of a Sterile Container Tray According to a Further embodiment.

The sterile container tray 10 is designed similarly to the sterile container tray 10 shown in FIG. 1a)-1c) or in FIG. 2a)-2c). The sterile container trays according to FIG. 1a)-1c) and FIG. 3a)-3c), or according to FIG. 2a)-2c) and FIG. 3a)-3c), differ in the contour of the tray rim 18.

In the example, the tray rim 18 in the first rim region 32 on the front rim portion 24 is formed with a curved contour, in particular continuously sloping towards the center of the end-face rim portion 24. Accordingly, the minimum region 36 in the example is a minimum point, i.e., the point with the lowest height of the tray rim 18.

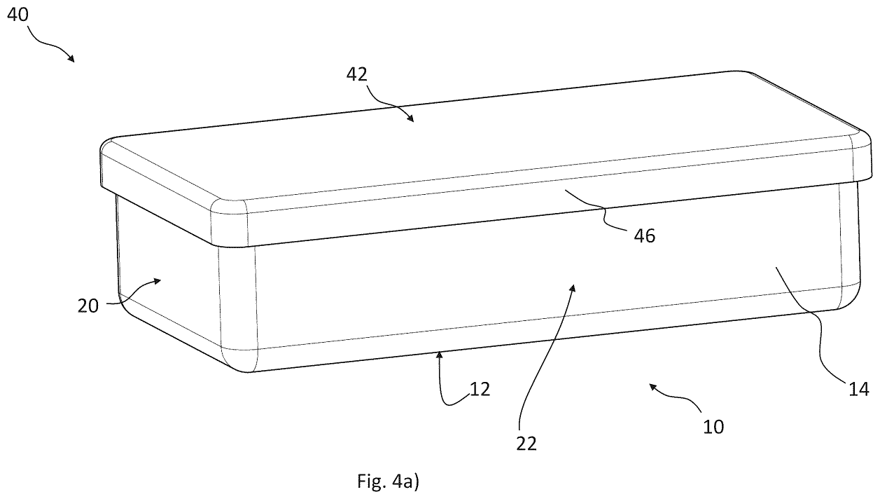

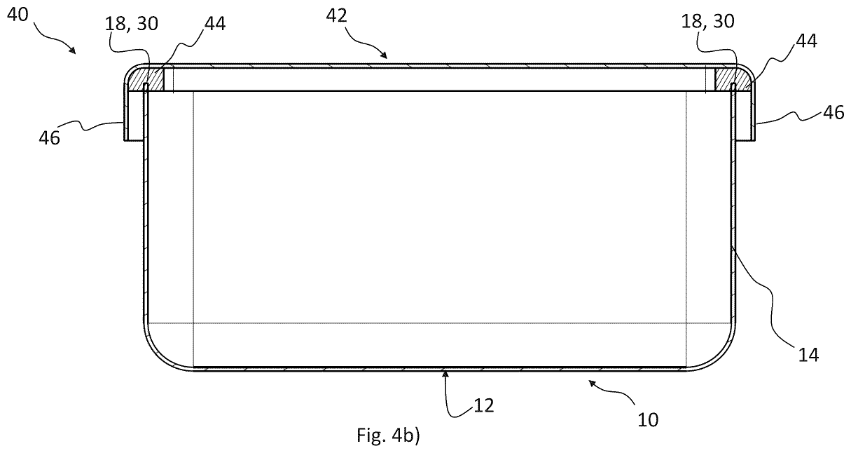

Finally, FIG. 4a) shows a sterile container 40. The sterile container 40 comprises a sterile container tray 10 which in the example can be designed in accordance with one of the embodiments in accordance with FIG. 1a)-3c). The sterile container 40 comprises a sterile container lid 42 for closing the sterile container tray 10 in the shown closed position. The sterile container 40 comprises, for example, a closing device (not shown), which is provided in particular on the sterile container tray 10 and/or on the sterile container lid 42 in order to clamp the sterile container lid 42 in the closed position against the sterile container tray 10 in a defined manner.

In this example, the sterile container lid 42 comprises a side rim 46 that overlaps the sterile container tray 10.

FIG. 4b) shows a sectional view of a portion through the sterile container 40 from FIG. 4a) transverse to the longitudinal side of the sterile container 40, i.e., a portion through the longitudinal wall portions 22 of the sterile container tray 10. The sectional view shows a sealing element 44 of the sterile container 40.

The sealing element 44 can, for example, be designed as a surrounding sealing lip. For example, the sealing element is arranged in the sterile container lid 42. In the closed position, the sealing element 44 lies against both the sterile container lid 42 and the sterile container tray 10, in the example, against the tray rim 18 of the sterile container tray 10.

When the sterile container lid 42 is clamped against the sterile container tray 10 in a defined manner by means of the closing device in the closed position, the sealing element 44 can be compressed. By means of the proposed height-varying contour of the tray rim 18, cf. FIG. 1a)-3c), deformation or bending of the sterile container lid 42 can be compensated for, and circumferential uniform contact of the sealing element 44 or uniform pressing of the sealing element 44 over the sterile container lid 42 onto the sterile container tray 10 can be ensured.

According to a further embodiment (not shown), it can be provided that the sealing element 44 in the closed position (only) lies against an outwardly directed region of the tray wall 14 (and not on the tray rim 18) of the sterile container tray 10.

### LIST OF REFERENCE NUMBERS

10 Sterile container tray
12 Tray bottom
14 Tray wall
16 Transition region
18 Tray rim
20 End-face wall portions
22 Longitudinal wall portions
24 End-face edge portions
26 Corners
28 Corner rim portion
30 Longitudinal rim portion
32 First rim region
34 Second rim region
36 Minimum region
38 Maximum region
40 Sterile container
42 Sterile container lid
44 Sealing element
46 Side rim

1. A sterile container tray for a sterile container, the sterile container tray comprising:
a tray bottom;
a tray wall surrounding the tray bottom; and
a tray rim forming a free end of the tray wall,
the tray rim comprising at least a first rim region and a second rim region,
the first rim region having a first height relative to the tray bottom, and
the second rim region having a second height relative to the tray bottom different from the first height.

2. The sterile container tray according to claim 1, wherein:
the first rim region comprises a minimum region at which the first height is at a minimum, and
the second rim region comprises a maximum region at which the second height is at a maximum.

3. The sterile container tray according to claim 1, wherein the tray wall comprises:
a first end-face wall portion;
a second end-face wall portion opposite the first end-face wall portion;
a first longitudinal wall portion; and
a second longitudinal wall portion opposite the first longitudinal wall portion,
wherein the first end-face wall portion and the second end-face wall portion each comprise an end-face rim portion,
wherein the first longitudinal wall portion and the second longitudinal wall portion each comprise a longitudinal rim portion,
wherein the first rim region is formed at least partially at each end-face rim portion, and
wherein the second rim region is formed at least partially at each longitudinal rim portion.

4. The sterile container tray according to claim 1, wherein the first rim region and/or the second rim region is formed by a contour of the tray rim that is at least in portions straight relative to the tray bottom.

5. The sterile container tray according to claim 1, wherein the first rim region and/or the second rim region is formed by a contour of the tray rim that continuously rises and continuously falls relative to the tray bottom.

6. The sterile container tray according to claim 1, wherein the first rim region and/or the second rim region is formed by a contour of the tray rim that is curved.

7. The sterile container tray according to claim 6, wherein the contour of the tray rim is at least in portions convex and/or concave.

8. The sterile container tray according to claim 1, wherein the tray wall comprises rounded corners, wherein a corner rim portion of the tray rim is formed at each of the rounded corners, and wherein the first rim region and/or the second rim region is formed at least partially at each corner rim portion.

9. The sterile container tray according to claim 1, wherein the tray rim comprises an outwardly curved profile.

10. The sterile container tray according to claim 1, wherein the tray wall defines a tray wall perimeter, and the tray rim extends from the tray wall in a height-varying manner along the tray wall perimeter.

11. The sterile container tray according to claim 1, wherein the tray wall extends perpendicularly to the tray bottom.

12. A sterile container comprising:
the sterile container tray according to claim 1;
a sterile container lid for closing the sterile container tray in a closed position; and
a sealing element.

13. The sterile container according to claim 12, wherein the sealing element lies against both the sterile container tray and the sterile container lid when the sterile container lid is in the closed position.

14. The sterile container according to claim 12, wherein the sealing element lies on the tray rim of the sterile container tray when the sterile container lid is in the closed position.

15. The sterile container according to claim 12, wherein the sealing element lies against an outwardly facing region of the tray wall when the sterile container lid is in the closed position.
